(19) United States
(12) Patent Application Publication
(10) Pub. No.: US 2024/0216504 A1
(43) Pub. Date: Jul. 4, 2024

(54) METHOD FOR PREVENTING OR TREATING OF HEMATHROSIS

(71) Applicants: Dong Kyu JIN, Seoul (KR); GREEN CROSS CORPORATION, Yongin (KR)

(72) Inventor: Dong Kyu JIN, Seoul (KR)

(73) Assignees: Dong Kyu JIN, Seoul (KR); GREEN CROSS CORPORATION, Yongin (KR)

(21) Appl. No.: 18/148,725

(22) Filed: Dec. 30, 2022

(51) Int. Cl. A61K 39/395; A61K 31/728; A61P 7/04; A61P 19/02

(52) U.S. Cl. CPC A61K 39/3955; A61K 31/728; A61P 7/04; A61P 19/02

(57) ABSTRACT

A pharmaceutical composition capable of exhibiting excellent efficacy against hemathrosis by including an anti-TFPI antibody and hyaluronic acid. A use of the pharmaceutical composition is also disclosed. A method for preventing or treating hemathrosis in a subject in need thereof, includes administering to the subject an effective amount of a pharmaceutical composition comprising an anti-TFPI antibody and hyaluronic acid.

### INCORPORATION BY REFERENCE OF SEQUENCE LISTING

The content of the electronically submitted sequence listing, file name: Q282260 Sequence_Listing_As_Filed.xml; size: 10,834 bytes; and date of creation: Dec. 28, 2022, filed herewith, is incorporated herein by reference in its entirety.

### BACKGROUND OF THE INVENTION

### 1. Field of the Invention

The present invention relates to a method for preventing or treating of hemathrosis.

### 2. Description of the Related Art

Hemathrosis is a phenomenon in which blood fills the joint cavity due to repeated intra-articular bleeding. Because the normal joint environment is broken, it leads to bad results.

A joint with repeated bleeding episodes is called a target joint, which typically means that about 4 individual bleeding occurred in the same joint over 6 months. However, the target joint can also be caused by severe bleeding.

The most common joints affected are the knees, ankles and elbows, but they can also occur on the hips, shoulders and wrists.

Hemathrosis is a common complication of hemophilia, but other causes include trauma due to sprains or injuries, side effects of blood thinners known as anticoagulants, joint infections, some types of arthritis such as osteoarthritis and hemophilic arthritis, some types of cancer, and leukemia in general.

Since the drug for hemathrosis has a weak effect and is invasive in the case of surgical treatment, it is necessary to develop an effective drug.

### SUMMARY OF THE INVENTION

It is an object of the present invention to provide a pharmaceutical composition for preventing or treating of hemathrosis.

It is an object of the present invention to provide a method for preventing or treating of hemathrosis

To achieve the above object, the following technical solutions are adopted in the present invention.
1. A pharmaceutical composition for preventing or treating of hemathrosis comprising an anti-TFPI antibody and hyaluronic acid.
2. The composition according to the above 1, wherein the anti-TFPI antibody comprises a heavy chain variable region comprising HCDR1 comprising the amino acid sequence of SEQ ID NO: 1, HCDR2 comprising the amino acid sequence of SEQ ID NO: 2, and HCDR3 comprising the amino acid sequence of SEQ ID NO: 3; and a light chain variable region comprising LCDR1 comprising the sequence of SEQ ID NO: 4, LCDR2 comprising the sequence of SEQ ID NO: 5, and LCDR3 comprising the sequence of SEQ ID NO: 6.
3. The composition according to the above 1, wherein the hyaluronic acid is sodium hyaluronate cross-linked with 1,4-butanediol diglycidyl ether (BDDE).
4. The composition according to the above 1, wherein the composition is an intra-articular injection formulation.

### BRIEF DESCRIPTION OF THE DRAWINGS

The above and other objects, features and other advantages of the present invention will be more clearly understood from the following detailed description taken in conjunction with the accompanying drawings, in which:

FIG. 1. The schematic diagram of preparing F8^{-/-} transgenic rabbits using CRISPR/Cas9.

FIGS. 2A and 2B. The Histologic findings in articular cartilage and synovium of the hemophilia A rabbits. (Hematoxylin and eosin stain. Original magnification: ×10, ×40, and ×100. Scale bar: 1000 μm (×10 panels), 200 μm (×40 panels), and 100 μm (×100 panels). FIGS. 2A and 2B are representative microphotographs of tissue cross-section of the joint cartilage of the rabbits in group A. There was less inflammation and hyperplasia of the joint surface in the treated group than in the control group. Most of the articular surfaces of the experimental group are intact except for minor erosion.

Black arrow: erosion or hyperplasia of synovial lining cell. Dotted arrow: osteolytic site in which inflammatory cells infiltrate the end of the cartilage surface and destroy the cortical bone. Open arrow: irregular and massive erosion on the cartilage surface. Asterix: artifact observed as chondrocytes are eliminated during the pathological tissue production process. UL: upper leg (elbow), LL: lower leg (knee).

FIGS. 3A-3C. The histologic scores between groups. In FIG. 3A, Cs: the histological score of cartilage changes. In FIG. 3B, Ss: the histological score of synovitis. In FIG. 3C, Os: the score of osteoarthritis, which indicates the combined score of Cs and Ss.

### DETAILED DESCRIPTION OF THE INVENTION

Hereinafter, the present invention will be described in detail. Unless otherwise specifically defined, all terms in the present specification would have the same meanings as general meanings of the corresponding terms understood by persons having common knowledge to which the present invention pertains (“those skilled in the art”), and if the general meanings conflict with the meanings of the terms used herein, the meanings used in the present specification take precedence.

The present invention relates to a pharmaceutical composition for preventing or treating of hemathrosis.

The pharmaceutical composition for preventing or treating of hemathrosis of the present invention includes an anti-TFPI antibody and hyaluronic acid.

Anti-TFPI antibody is an anti-tissue factor pathway inhibitor (hereinafter referred to as anti-TFPI) recombinant antibody which has high affinity for a tissue factor pathway coagulation inhibitor (hereinafter referred to as TFPI).

Anti-TFPI antibody inhibits the binding between TFPI and activated factor X (FXa) and induces the activation of the lower coagulation factor FX in the extrinsic coagulation pathway, thereby being used as a treatment for hemophilia.

In the present invention, as an anti-TFPI antibody, as long as it can bind to TFPI, its type, detailed sequence, etc. are not limited, and those known in the art may be used. For example, as an anti-TFPI antibody, one described in Korean Patent No. 1804988, a known antibody MG1113, and the like can be used.

In the present invention, an anti-TFPI antibody, for example, may comprises a heavy chain variable region comprising HCDR1 comprising the amino acid sequence of SEQ ID NO: 1, HCDR2 comprising the amino acid sequence of SEQ ID NO: 2, and HCDR3 comprising the amino acid sequence of SEQ ID NO: 3; and a light chain variable region comprising LCDR1 comprising the sequence of SEQ ID NO: 4, LCDR2 comprising the sequence of SEQ ID NO: 5, and LCDR3 comprising the sequence of SEQ ID NO: 6. The entire sequence of the heavy chain variable region of the anti-TFPI antibody may comprise the amino acid sequence of SEQ ID NO: 7. The entire sequence of the light chain variable region of the anti-TFPI antibody may comprise the amino acid sequence of SEQ ID NO: 8.

In the present invention, an anti-TFPI antibody may be included in, for example, 0.0001% to 10% of the total weight of the composition.

Hyaluronic acid (HA) is a natural substance with biocompatibility that is abundant in animal skin, joint fluid, and cartilage. It is a component constituting the matrix in articular cartilage and is a kind of mucopolysaccharide involved in making proteoglycan. It is a glycoprotein complex of N-acetyl-D-glucosamine and D-glucuronic acid linked by 1-4 glycosidic bonds. Hyaluronic acid can be combined with water to exist in a gel state.

In the present invention, hyaluronic acid may be cross-linked through a physical method such as ultraviolet rays, radiation, electron beams, or a chemical method using 1,4-butanediol diglycidyl ether (BDDE).

In the present invention, hyaluronic acid may be included as a pharmaceutically acceptable salt. Pharmaceutically acceptable salts of hyaluronic acid include, for example, both inorganic salts such as sodium hyaluronate, magnesium hyaluronate, zinc hyaluronate and cobalt hyaluronate, and organic salts such as tetrabutylammonium hyaluronate. In some cases, a combination of at least two of these may be used.

In the present invention, the molecular weight of hyaluronic acid is not particularly limited, it is preferably, for example, an average molecular weight of 500,000 to 10,000,000.

In the present invention, hyaluronic acid may be included, for example, from 0.0001% to 10% by weight of the total weight of the composition.

Hemathrosis is a phenomenon in which blood fills the joint due to repeated intra-articular bleeding. Because the normal joint environment is broken, warmth, edema, and pain occur in the beginning, and symptoms can become serious over time, resulting in swelling, joint stiffness, pain, loss of exercise, and discomfort, and permanent damage to the joints. Joint damage may occur in a form similar to arthritis in the form of cartilage destruction/erosion.

In the present invention, a subject to application of the composition of the present invention is a subject that may develop hemathrosis or has the same. It can be a mammal, including a human being, or a human being.

In the present invention, the pharmaceutical composition of the present invention may be in the form of a capsule, tablet, granule, injection, ointment, powder, or beverage.

In the present invention, the pharmaceutical composition of the present invention is not limited thereto, but may be formulated and used in the form of an oral formulation such as an powder, a granule, a capsule, a tablet, an aqueous suspension, and an external preparation, a suppository and a sterilizing injection solution according to a conventional method. The pharmaceutical composition of the present invention may include a pharmaceutically acceptable carrier. For oral administration, pharmaceutically acceptable carriers may include a binder, a lubricant, a disintegrant, an excipient, a solubilizer, a dispersant, a stabilizer, a suspending agent, a coloring agent, a flavoring agent, and the like. In the case of an injection, a buffer, a preservative, an analgesic, a solubilizer, an isotonic agent, a stabilizer, and the like may be mixed and used. For topical administration, a substrate, an excipient, a lubricant, a preservative, and the like may be used. The formulation of the pharmaceutical composition of the present invention may be prepared in various ways by mixing with the pharmaceutically acceptable carrier as described above. For example, in oral administration, it can be prepared in the form of tablets, troquis, capsules, elixir, suspension, syrup, wafers, etc., and in the case of injections, it can be prepared in the form of a unit dose ampoule or multiple doses. It can be formulated as solutions, suspensions, tablets, capsules, and sustained formulations, etc.

In the present invention, examples of carriers, excipients, and diluents suitable for formulation include lactose, dextrose, sucrose, solvitol, mannitol, xylitol, erythritol, malditol, starch, acacia rubber, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate, or mineral oil, and the like. In addition, a filler, an anticoagulant, a lubricant, a wetting agent, a fragrance, an emulsifier, a preservative, and the like may be further included.

In the present invention, the administration route of the pharmaceutical composition of the present invention is not limited to them, but includes, oral, intravenous, intramuscular, intraarterial, intramedullary, intrathecal, intracardiac, transdermal, subcutaneous, intraperitoneal, intranasal, enteral, topical, sublingual, or rectal. Oral or parenteral administration is preferred.

In the present invention, parenteral includes subcutaneous, intradermal, intravenous, intramuscular, intraarticular, intrasynovial, intrasternal, intrathecal, intralesional and intracranial injection or infusion techniques. The pharmaceutical composition of the present invention may also be administered in the form of a suppository for rectal administration. Specifically, the pharmaceutical composition of the present invention may be in the form of intra-articular administration, and more specifically, it may be an intra-articular injection.

In the present invention, the pharmaceutical composition of the present invention may vary depending on various factors including the activity of the specific compound used, age, body weight, general health, sex, diet, administration time, route of administration, excretion rate, drug combination and severity of the specific disease to be prevented or treated. The dosage of the pharmaceutical composition may vary depending on the patient's condition, body weight, degree of disease, drug form, administration route and period, but can be appropriately selected by those skilled in the art. For example, it may be administered at 0.0001 to 50 mg/kg or 0.001 to 50 mg/kg per day. It may be administered once a day or divided into several doses. The dosage is not intended to limit the scope of the present invention in any way. The pharmaceutical composition according to the present invention may be formulated into a pill, dragee, capsule, liquid, gel, syrup, slurry, or suspension.

In addition, the present invention relates to a method for treating of hemathrosis.

The method of treating of hemathrosis of the present invention comprises administering to a subject a composition comprising an anti-TFPI antibody and hyaluronic acid.

The subject is a subject in need of treatment for hemathrosis. It may be a mammal, including a human, and may be a human.

As the anti-TFPI antibody, those within the above range may be used.

Hyaluronic acid may be used within the above range.

A composition comprising an anti-TFPI antibody and hyaluronic acid may be used within the above range.

Administration can be performed by routes known in the art.

In the present invention, routes of administration include, but are not limited to, oral, intravenous, intramuscular, intraarterial, intramedullary, intrathecal, intracardiac, transdermal, subcutaneous, intraperitoneal, intranasal, enteral, topical, sublingual, or rectal. Oral or parenteral administration is preferred.

In the present invention, parenteral includes subcutaneous, intradermal, intravenous, intramuscular, intraarticular, intrasynovial, intrasternal, intrathecal, intralesional and intracranial injection or infusion techniques. The pharmaceutical composition of the present invention may also be administered in the form of a suppository for rectal administration. Specifically, intra-articular administration may be performed, and more specifically, intra-articular injection may be used.

In the present invention, the dosage of the composition may be appropriately selected by those skilled in the art, although it varies depending on the patient's condition, body weight, degree of disease, drug form, administration route and period. For example, it may be administered at 0.0001 to 50 mg/kg or 0.001 to 50 mg/kg per day. It may be administered once a day or divided into several doses. The dosage is not intended to limit the scope of the present invention in any way.

Hereinafter, the present invention will be described in detail with reference to the following examples.

### Method

### Animal Welfare

This study has been approved by the Institutional Animal Care and Use Committee and performed by the animal welfare policy of Samsung Biomedical Research Institute (SBRI).

### CRISPR/Cas9-Mediated FVIII Knockout Transgenic Rabbit Preparation

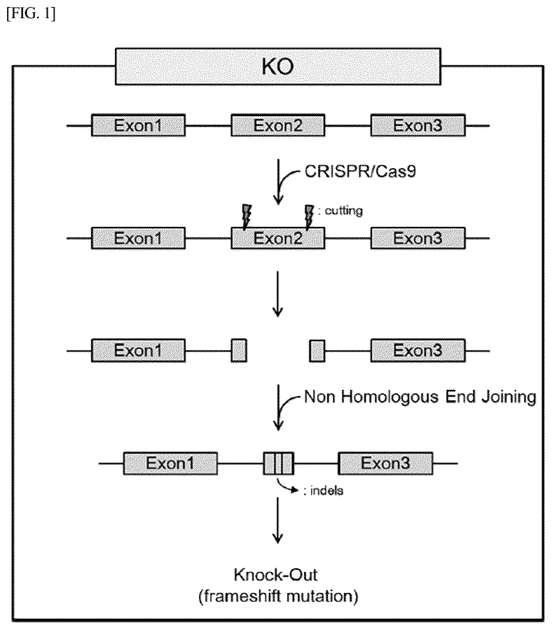

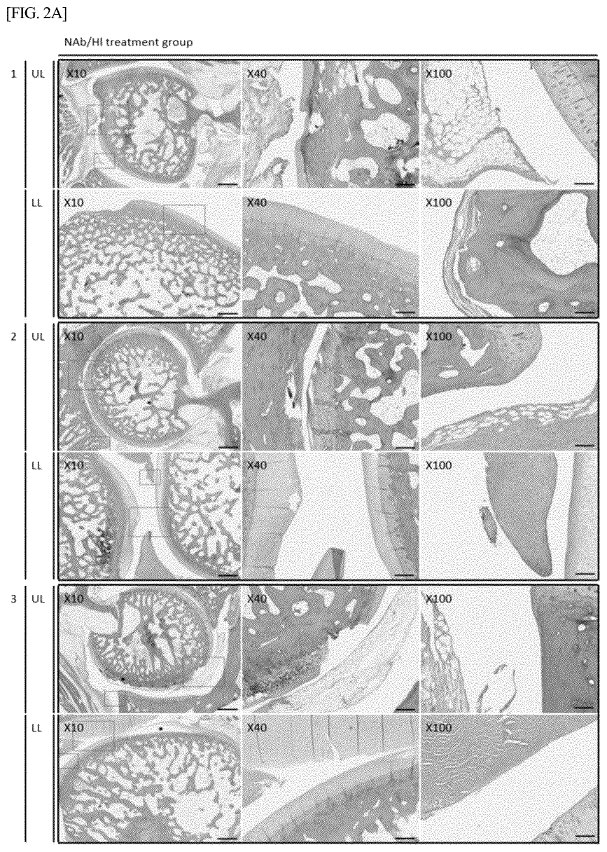

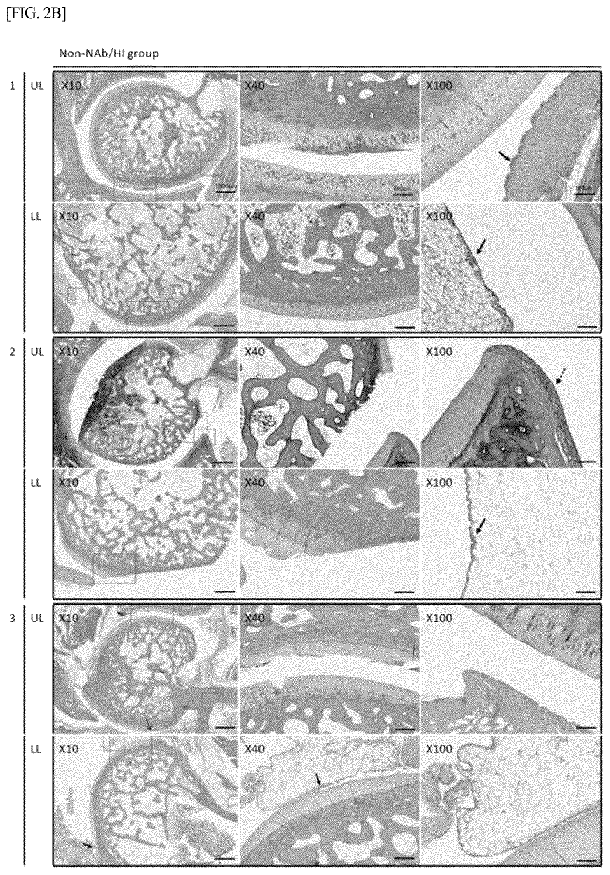

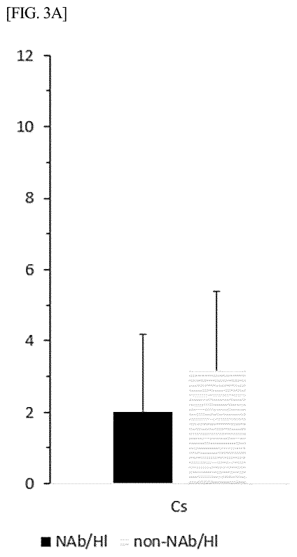

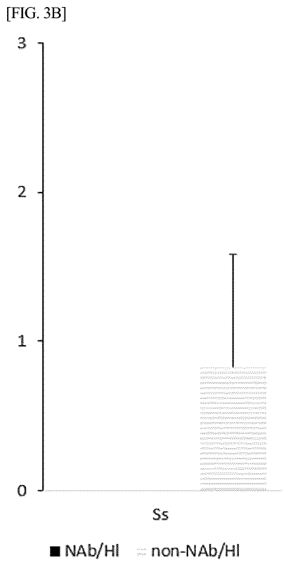

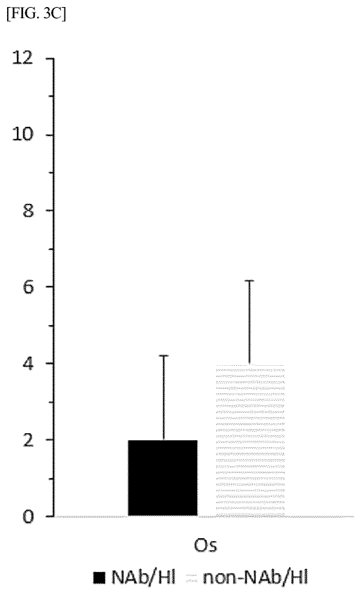

This study is an open-labeled, controlled monocentric study, using New Zealand white rabbit with factor VIII knockout (F8^{-/-}) diagnosed with phenotypically proven hemophilia A aged more than five to nine months. The rabbit is a suitable model for osteoarthritis and yields an adequate amount of tissue for a comprehensive histopathological assessment of structural changes associated with the disease process. A total of three transgenic rabbits with F8^{-/-} using the CRISPR/Cas9 system were prepared (FIG. 1). Transgenic rabbit males (P) whose genotype and phenotype were confirmed were crossed with some normal rabbit females to generate offspring. The offspring females (F1) were genetically analyzed to identify carriers and crossed with normal males to produce offspring. Transgenic rabbit males (F2) were obtained through this process. The rabbits were housed in a cage under a 12-hour light-dark cycle at a temperature of 24-26° C. with access to water and food ad libitum at the animal testing facilities. The animals were bred and kept in the Laboratory in GC Biopharma (Yong-in, Korea).

### Phenotype Confirmation

Hemarthrosis was suspected clinically as all F2 rabbits showed bleeding, edema, and pain around the joints from the age of 6 weeks. When bleeding-related symptoms occurred, factor VIII (GreenGene F®, GC Biopharma, Yongin, Korea) was administered intravenously to improve symptoms. The phenotype of F2 was confirmed using the rabbit cuticle bleeding test (CBT). This in vivo hemostasis test has been previously validated in a canine model of novel prohemostatic agents and anticoagulants. Briefly, 10 minutes after anesthetic induction, the rabbits were placed in the lateral recumbency position, and all hair around the nail bed was carefully removed by clipping around the base of the claw. The same experienced veterinary technologist performed all CBT assays.

### MG1113 Mixture Preparation and Administration

One unilateral side of the upper and lower limb was used for the experiment group. The contralateral control group of the limb remained naïve. All intra-articular injections were applied under intravenous anesthesia by weight-counted pentobarbital (ENTOBAR® INJ, 50 mg/mL, Hanlim Pharm. co., ltd., Korea) using a 3 ml plastic syringe and a 27 G needle with a capacity of 0.6 mL/kg for pain relief. The animal's leg and arm were placed on the edge of the operating table and held in place by an assistant. Both groups received two injections of MG1113 mixture four weeks apart or remained naïve. One experienced technician performed all drugs, adept at injecting into the joints of dead rabbits. The first injection was done the day 1 on week 1 and repeated with consecutive injections on day 29 on week 5. Each injection was administered after an intravenous MG1113 infusion 24 hours before at 10 mg/kg. The group A (NAb/Hl: NAb mixed with Hl injection group, n=12) received 1 ml of intra-articular Hl at a dose of 10 mg/mL (MW=2×10⁶ Da, SYNOVIAN® INJ, 10 mg/mL, LG Corp., Korea) mixed with NAb at a dose of 16.67 mg/mL (MG1113, 115.4 mg/ml) into the right-sided elbow and knee joints leaving clear on the left side as a control group (non-NAb/Hl).

The rabbits regained consciousness about 30 minutes after administering the anesthetic injection and entered the cage in a stable state. During the whole experiment, the animals could move around freely, reach the food pellets, and drink the nozzle in the corner of their cage. Animals were euthanized at day 57 on week 9, and knee joints were harvested for histological analysis.

### Histological Analyses

The joints were removed on day 35 and fixed for 24 h in 4% paraformaldehyde. The fixed tissues were decalcified in Decalcifying Solution, Formic Acid 5%, Aqueous (Newcomer Supply, Inc., Wisconsin, USA) for one month, embedded in paraffin and sectioned (4 μm) using a microtome. Tissue sections were stained with hematoxylin and eosin (H&E) staining to assess synovial inflammation and cartilage damage.

H&E-stained sections were analyzed for cartilage damage using the inverse grading system. Briefly, this score involves the histologic evaluation of cartilage change, synovitis, and osteoarthritis. The histologic score of cartilage change (Cs) ranges from 0 for normal cartilage to 11 for cartilage with full-depth hyaline erosion and calcified cartilage to the subchondral bone being ≥50% articular surface. The histologic score of synovitis (Ss) ranges from 0 to 3, which measures the severity of synovitis. The histologic score of osteoarthritis (Os), which indicates the summation of the previous two scores for each compartment, was also analyzed. Pathological analysis was performed on 24 joint surfaces from six F8^{-/-} rabbits at week 9. One blinded investigator gave the scores.

### Statistical Analysis

Data are presented as mean values: 95% confidence intervals were used to denote the statistical uncertainty of the estimates. Data were analyzed with the SPSS software for Windows, version 25.0 (SPSS, Chicago. IL). Correlations were significant if p-values were <0.05. Shapiro-Wilk's normality test was performed to verify whether the data collected for this study satisfied the normality assumption (P>0.05). Data are expressed as the mean±standard error of the mean (SEM) or median and interquartile range (IQR), as appropriate. Student's t-test was used to compare two independent groups for normally distributed parameters. The Mann-Whitney U-test was used to compare two independent groups for non-normally distributed parameters. Bonferroni corrections were performed to determine statistical significance.

### Results

Each test condition was performed with n=3 independent animals (n=12, articular plates of upper or lower limbs). These are likely correlated as the data involve multiple observations on different joints from the same animals.

There was no significant difference in the weight distribution between groups from pre/post-injection (Table 1). Throughout the study, the rabbits showed no pain or restriction of joint movement and moved freely. Their food and water consumption were not reduced. The stress of the participating animals by injection was not noticeable.

TABLE 1

| | | Before 2 weeks* | | First injection** | | Second injection** | |
|---|---|---|---|---|---|---|---|
| | No. | Age (months) | Weight (kg) | Age (months) | Weight (kg) | Age (months) | Weight (kg) |
| Group A | 1 | 9 | 4.05 | 9 | 4 | 10 | 3.95 |
| (n = 3) | 2 | 8 | 3.5 | 8 | 3.4 | 9 | 3.5 |
| | 3 | 8 | 3.6 | 8 | 3.45 | 9 | 3.5 |
| Average *** | | | 3.72 | | 3.62 | | 3.65 |

Grossly, the NAb/Hl treated group showed less inflammation and hyperplasia of the joint surface than the non-NAb/Hl group on the H&E stain. Most of the articular surfaces of the treated group are intact except for minor erosion in group A. The results of the histologic score were described in table 2.

TABLE 2

| | NAb/Hl | Non-NAb/Hl |
|---|---|---|
| Cs | 2.00 ± 2.19 (2.00) | 3.17 ± 2.23 (4.00) |
| Ss | 0.00 ± 0.00 (0.00) | 0.83 ± 0.75 (1.00) |
| Os | 2.00 ± 2.19 (2.00) | 4.00 ± 2.19 (4.50) |

Referring to Table 2, it can be confirmed that the NAb/HI mixture treatment group has lowered all of Cs, Ss, and Os compared to the untreated group, and thus the tissue within the joint is improved.

What is claimed is:

1. A pharmaceutical composition for preventing or treating of hemathrosis comprising an anti-TFPI antibody and hyaluronic acid.

2. The composition according to claim 1, wherein the anti-TFPI antibody comprises a heavy chain variable region comprising HCDR1 comprising the amino acid sequence of SEQ ID NO: 1, HCDR2 comprising the amino acid sequence of SEQ ID NO: 2, and HCDR3 comprising the amino acid sequence of SEQ ID NO: 3: And a light chain variable region comprising LCDR1 comprising the sequence of SEQ ID NO: 4, LCDR2 comprising the sequence of SEQ ID NO: 5, and LCDR3 comprising the sequence of SEQ ID NO: 6.

3. The composition according to claim 1, wherein the hyaluronic acid is sodium hyaluronate cross-linked with 1,4-butanediol diglycidyl ether (BDDE).

4. The composition according to claim 1, wherein the composition is an intra-articular injection formulation.

5. A method for preventing or treating hemathrosis in a subject in need thereof, comprising administering to the subject an effective amount of a pharmaceutical composition comprising an anti-TFPI antibody and hyaluronic acid.

6. The method according to claim 5, wherein the anti-TFPI antibody comprises a heavy chain variable region comprising HCDR1 comprising the amino acid sequence of SEQ ID NO: 1, HCDR2 comprising the amino acid sequence of SEQ ID NO: 2, and HCDR3 comprising the amino acid sequence of SEQ ID NO: 3: And a light chain variable region comprising LCDR1 comprising the sequence of SEQ ID NO: 4, LCDR2 comprising the sequence of SEQ ID NO: 5, and LCDR3 comprising the sequence of SEQ ID NO: 6.

7. The method according to claim 5, wherein the hyaluronic acid is sodium hyaluronate cross-linked with 1,4-butanediol diglycidyl ether (BDDE).

8. The method according to claim 5, wherein the composition is administered via an intra-articular injection route.


SEQUENCE LISTING (8 sequences)

SEQ ID NO: 1 - protein, 5 aa, synthetic construct
  REGION 1..5: note = anti TFPI antibody HCDR1
SYAMN

SEQ ID NO: 2 - protein, 17 aa, synthetic construct
  REGION 1..17: note = anti TFPI antibody HCDR2
TITTGGSYTYYADSVDG

SEQ ID NO: 3 - protein, 9 aa, synthetic construct
  REGION 1..9: note = anti TFPI antibody HCDR3
QDGNFLMDY

SEQ ID NO: 4 - protein, 16 aa, synthetic construct
  REGION 1..16: note = anti TFPI antibody LCDR1
KSSQSLLDIDGKTYLN

SEQ ID NO: 5 - protein, 7 aa, synthetic construct
  REGION 1..7: note = anti TFPI antibody LCDR2
LVSKLDS

SEQ ID NO: 6 - protein, 8 aa, synthetic construct
  REGION 1..8: note = anti TFPI antibody LCDR3
WQGTHFPF

SEQ ID NO: 7 - protein, 118 aa, synthetic construct
  REGION 1..118: note = anti TFPI antibody H chain variable region
EVQLVESGGGLVKPGGSLRLSCAASGFTFSSYAMNWVRQAPGKGLEWVSTITTGGSYTYYADSVDGRFTISRDNAKNSLYLQMNSLRAEDTAVYYCARQDGNFLMDYWGQGTLVTVSS

SEQ ID NO: 8 - protein, 113 aa, synthetic construct
  REGION 1..113: note = anti TFPI antibody L chain variable region
DVMMTQTPLSLPVTLGQPASISCKSSQSLLDIDGKTYLNWLQQRPGQSPKRLIYLVSKLDSGVPDRFTGSGSGTDFALKISRVEAEDVGVYYCWQGTHFPFTFGQGTKVEIKR